(19) United States
(12) Patent Application Publication
(10) Pub. No.: US 2006/0148828 A1
(43) Pub. Date: Jul. 6, 2006

(54) Combination comprising a CDK inhibitor and a topoisomerase 1 inhibitor for the treatment of cancer and other proliferative diseases

(75) Inventors: Athos Gianella-Borradori, Gauting (DE); Marie-France Poupon, Paris (FR)

Correspondence Address: LAHIVE & COCKFIELD, 28 STATE STREET, BOSTON, MA, 02109, US

(73) Assignee: Cyclacel Limited, Dundee (GB)

(21) Appl. No.: 11/301,070

(22) Filed: Dec. 9, 2005

Related U.S. Application Data
(63) Continuation of application No. PCT/GB04/02446, filed on Jun. 10, 2004.

(30) Foreign Application Priority Data
Jun. 11, 2003 (GB) 0313511.8

(51) Int. Cl. A61K 31/522; A61K 31/4745

(52) U.S. Cl. 514/263.32; 514/283

(57) ABSTRACT

A first aspect of the invention relates to a combination comprising a CDK inhibitor and CPT-11. A second aspect of the invention relates to a pharmaceutical product comprising a CDK inhibitor and CPT-11 as a combined preparation for simultaneous, sequential or separate use in therapy. A third aspect of the invention relates to a method of treating a proliferative disorder, said method comprising simultaneously, sequentially or separately administering a CDK inhibitor and CPT-11 to a subject.

### RELATED APPLICATIONS

This application is a continuation of PCT/GB2004/002446, filed Jun. 10, 2004, which claims priority to GB Application No. 0313511.8, filed Jun. 11, 2003. The entire contents of each of these applications is hereby expressly incorporated by reference herein.

### FIELD OF THE INVENTION

The present invention relates to a pharmaceutical combination suitable for the treatment of cancer and other proliferative disorders.

### BACKGROUND TO INVENTION

Initiation, progression, and completion of the mammalian cell cycle are regulated by various cyclin-dependent kinase (CDK) complexes, which are critical for cell growth. These complexes comprise at least a catalytic (the CDK itself) subunit and a regulatory (cyclin) subunit. Some of the more important complexes for cell cycle regulation include cyclin A (CDK1-also known as cdc2, and CDK2), cyclin B1-B3 (CDK1), cyclin C (CDK8), cyclin D1-D3 (CDK2, CDK4, CDK5, CDK6), cyclin E (CDK2), cyclins K and T (CDK9) and cyclin H (CDK7). Each of these complexes is involved in a particular phase of the cell cycle.

The activity of CDKs is regulated post-translationally, by transitory associations with other proteins, and by alterations of their intracellular localisation. Tumour development is closely associated with genetic alteration and deregulation of CDKs and their regulators, suggesting that inhibitors of CDKs may be useful anti-cancer therapeutics. Indeed, early results suggest that transformed and normal cells differ in their requirement for e.g. cyclin A/CDK2 and that it may be possible to develop novel antineoplastic agents devoid of the general host toxicity observed with conventional cytotoxic and cytostatic drugs.

The function of CDKs is to phosphorylate and thus activate or deactivate certain proteins, including, for example, retinoblastoma proteins, lamins, histone Hi, and components of the mitotic spindle. The catalytic step mediated by CDKs involves a phospho-transfer reaction from ATP to the macromolecular enzyme substrate. Several groups of compounds (reviewed in N. Gray, L. Détivaud, C. Doerig, L. Meijer, Curr. Med. Chem. 1999, 6, 859) have been found to possess anti-proliferative properties by virtue of CDK-specific ATP antagonism.

Roscovitine is the compound 6-benzylamino-2-[(R)-1-ethyl-2-hydroxyethylamino]-9-isopropylpurine. Roscovitine has been demonstrated to be a potent inhibitor of cyclin dependent kinase enzymes, particularly CDK2. This compound is currently in development as an anti-cancer agent. CDK inhibitors are understood to block passage of cells from the G1/S and the G2/M phase of the cell cycle. Roscovitine has also been shown to be an inhibitor of retinoblastoma phosphorylation and therefore implicated as acting more potently on Rb positive tumors.

It is well established in the art that active pharmaceutical agents can often be given in combination in order to optimise the treatment regime. The present invention therefore seeks to provide a new combination of known pharmaceutical agents that is particularly suitable for the treatment of proliferative disorders, especially cancer. More specifically, the invention centres on the surprising and unexpected effects associated with using certain pharmaceutical agents in combination.

### STATEMENT OF INVENTION

In a first aspect, the invention provides a combination comprising a CDK inhibitor and CPT-11.

A second aspect provides a pharmaceutical composition comprising a combination according to the invention admixed with a pharmaceutically acceptable carrier, diluent or excipient.

A third aspect relates to the use of a combination according to the invention in the preparation of a medicament for treating a proliferative disorder.

A fourth aspect relates to a pharmaceutical product comprising a CDK inhibitor and CPT-11 as a combined preparation for simultaneous, sequential or separate use in therapy

A fifth aspect relates to a method of treating a proliferative disorder, said method comprising simultaneously, sequentially or separately administering a CDK inhibitor and CPT-11 to a subject.

A sixth aspect relates to the use of a CDK inhibitor in the preparation of a medicament for the treatment of a proliferative disorder, wherein said treatment comprises simultaneously, sequentially or separately administering a CDK inhibitor and CPT-11 to a subject.

A seventh aspect relates to the use of a CDK inhibitor and CPT-11 in the preparation of a medicament for treating a proliferative disorder.

An eighth aspect relates to the use of a CDK inhibitor in the preparation of a medicament for the treatment of a proliferative disorder, wherein said medicament is for use in combination therapy with CPT-11.

A ninth aspect relates to the use of CPT-11 in the preparation of a medicament for the treatment of a proliferative disorder, wherein said medicament is for use in combination therapy with a CDK inhibitor.

A tenth aspect of the invention relates to a combination comprising a CDK inhibitor and a DNA topoisomerase 1 inhibitor.

### DETAILED DESCRIPTION

The preferred embodiments as set out below are applicable to all the above-mentioned aspects of the invention.

As mentioned above, the present invention relates to a combination comprising a CDK inhibitor and CPT-11.

CPT-11, also know as irinotecan, is a DNA topoisomerase 1 inhibitor that induces double strand breaks. CPT-11 is a semisynthetic derivative of camptothecin, converted in vivo into its active form SN-38, with cytotoxic effects exerted through its binding to and inhibition of the DNA-associated nuclear enzyme topoisomerase 1 (top1), thus stabilizing top1 DNA cleavable ternary complexes (5). This impedes the DNA-religation reaction and results in DNA double-strand breaks, eventually leading to apoptosis (6).

The effect of drug combinations is inherently unpredictable and there is often a propensity for one drug to partially or completely inhibit the effects of the other. The present invention is based on the surprising observation that administering CPT-11 and a CDK inhibitor (for example, roscovitine) in combination, either simultaneously, separately or sequentially, does not lead to any adverse interaction between the two agents. The unexpected absence of any such antagonistic interaction is critical for clinical applications.

Preferably, the combination has a synergistic effect, i.e. the combination is synergistic.

As mentioned above, one aspect of the invention relates to a pharmaceutical product comprising a CDK inhibitor and CPT-11 as a combined preparation for simultaneous, sequential or separate use in therapy.

The CDK inhibitor and CPT-11 may be administered simultaneously, in combination, sequentially or separately (as part of a dosing regime).

As used herein, “simultaneously” is used to mean that the two agents are administered concurrently, whereas the term “in combination” is used to mean they are administered, if not simultaneously, then “sequentially” within a timeframe that they both are available to act therapeutically within the same time-frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction therebetween, and their respective half-lives.

In contrast to “in combination” or “sequentially”, “separately” is used herein to mean that the gap between administering one agent and the other is significant i.e. the first administered agent may no longer be present in the bloodstream in a therapeutically effective amount when the second agent is administered.

One aspect of the present invention relates to the use of a CDK inhibitor in the preparation of a medicament for the treatment of a proliferative disorder, wherein said treatment comprises administering to a subject simultaneously, sequentially or separately CPT-11 and a CDK inhibitor.

Preferably, the CDK inhibitor and CPT-11 are administered simultaneously or sequentially.

In one preferred embodiment, the CPT-11 and CDK inhibitor are administered simultaneously.

In one particularly preferred embodiment, the CDK inhibitor is administered to the subject prior to sequentially or separately administering CPT-11 to said subject.

Another aspect of the invention relates to a method of treating a proliferative disorder comprising the sequential administration of a therapeutically effective amount of CDK inhibitor followed by a therapeutically effective amount of CPT-11. Another aspect of the invention relates to the use of roscovitine in the manufacture of a medicament for use in the treatment of proliferative disorders comprising the sequential administration of a therapeutically effective amount of CDK inhibitor followed by a therapeutically effective amount of CPT-11.

In an alternative preferred embodiment, CPT-11 is administered to the subject prior to sequentially or separately administering a CDK inhibitor to said subject.

In one particularly preferred embodiment, the CDK inhibitor and CPT-11 are administered sequentially.

In one preferred embodiment of the invention, the CDK inhibitor and CPT-11 are each administered in a therapeutically effective amount with respect to the individual components.

In another preferred embodiment of the invention, the CDK inhibitor and CPT-11 are each administered in a subtherapeutic amount with respect to the individual components.

In another preferred embodiment of the invention, the CPT-11 is administered in an amount sufficient to cause an increase in CDK1 levels.

In yet another preferred embodiment of the invention, the CDK inhibitor is administered in an amount sufficient to induce apoptosis.

In one particularly preferred embodiment of the invention, the CPT-11 is administered on days 1, 8, and 5, and the CDK inhibitor is administered on days 2-5, 9-12 and 16-19. Preferably, the CDK inhibitor is roscovitine.

Preferably, the roscovitine is administered in an amount of about 200 to about 500 mg/kg/day, more preferably about 300 to about 400 mg/kg/day. More preferably, the roscovitine is administered in an amount of about 300 to about 400 mg/kg/day divided into two doses, i.e. 2×150-200 mg/kg doses. Preferably, the two daily doses are separated by 6 to 8 hours.

Preferably, the roscovitine is administered orally.

Preferably, the CPT-11 is administered in an amount of about 20 to about 50 mg/kg/day, more preferably about 25 to about 45 mg/kg/day, more preferably still about 30 to about 40 mg/kg/day, even more preferably about 40 mg/kg/day.

Preferably, the CPT-11 is administered by intraperitoneal route.

Another aspect of the invention relates to the use of a CDK inhibitor and CPT-11 in the preparation of a medicament for treating a proliferative disorder.

Yet another aspect of the invention relates to the use of a CDK inhibitor in the preparation of a medicament for the treatment of a proliferative disorder, wherein said medicament is for use in combination therapy with CPT-11.

A further aspect of the invention relates to the use of CPT-11 in the preparation of a medicament for the treatment of a proliferative disorder, wherein said medicament is for use in combination therapy with a CDK inhibitor.

As used herein, the term “combination therapy” refers to therapy in which the CPT-11 and CDK inhibitor are administered, if not simultaneously, then sequentially within a timeframe that they both are available to act therapeutically within the same time-frame.

As used herein the phrase “preparation of a medicament” includes the use of the components of the invention directly as the medicament in addition to their use in any stage of the preparation of such a medicament.

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the components of the present invention may induce apoptosis or maintain stasis within the desired cells as required.

Preferably, the proliferative disorder is a cancer or leukaemia, most preferably cancer.

Where the proliferative disorder is cancer, the cancer may be a p53-dependent or p53-independent cancer.

In one particularly preferred embodiment, the proliferative disorder is a p53-independent cancer.

In one especially preferred embodiment, the proliferative disorder is colorectal cancer, more preferably colon cancer.

In another preferred embodiment, the proliferative disorder is lung cancer.

Colorectal cancer (CRC) is the second leading cause of cancer death in Western countries, and not withstanding the efforts made to improve chemotherapy, response rates have not been associated with a significant survival benefit. For many years, standard therapy for advanced CRC has been based on the thymidylate-synthase inhibitor 5-fluorouracil (5-FU). Recently, new compounds with different mechanisms of action have demonstrated increased response rates (1). CPT-11, as a single agent, showed tumor response in patients with 5-FU resistant CRC (2). CPT-11 has been approved, in combination with 5-FU and the modulator leucovorin, as first-line chemotherapy for patients with metastatic CRC (3, 4). However, to date there has been no suggestion of administering CPT-11 in combination with a CDK inhibitor such as roscovitine.

Advanced CRC is known to involve mutations in the tumor suppressor gene p53. CPT-11 has been recently incorporated to the adjuvant therapy, which is crucial at advanced stages of the disease. Since the DNA-damage checkpoint depends on p53 activation, the status of p53 might critically influence the response to CPT-11.

Sensitivity to CPT-11 may depend on top 1 activity, associated deficiencies in DNA repair and cell cycle regulation, and on the inability of cancer cells to repress apoptosis. In this context, the influence of the p53 status to the response of tumor cells to CPT-11 remains controversial. Firstly, p53 would contribute by protecting cells against CPT-11-induced damage, as shown by the correlation of CPT-11 with long-term arrest in the p53+/+ HCT116 colorectal carcinoma cell line, and with apoptosis in the p53−/− knocked-out derived HCT116 cell line (7). In addition, increased cytotoxicity was observed in MCF-7 breast carcinoma and HCT116 cells upon p53 inactivation (8). Secondly, p53 would sensitize cells to CPT-11, as described in a variety of human cancer cell lines and normal human fibroblasts (9). In vivo studies with xenografted human colorectal cancers have shown that mutated p53 status correlated with a poor response to CPT-11(10), and with significant lower levels of DNA topoisomerase I complexes trapped by camptothecin (11). Finally, the combination of irradiation and SN-38 treatment showed supraadditive effects on fibroblasts, independently of the p53 status (12).

To better understand the involvement of p53, the sensitivity to CPT-11 was compared, both in vivo and in vitro, in the mut-p53 HT29 colon cancer cell line and the wt-p53 subclone HT29-A4. Further details of these studies may be found in the accompanying Examples.

By way of sumnmary, cell cycle analysis after treatment with CPT-11 in G0/G1 synchronized cells demonstrated the activation of transfected wild-type p53 and a consequent p^{21WAF1/CIP1}-dependent cell cycle blockage in S phase. Activated wt-p53 also increased apoptosis, leading to enhanced sensitivity to CPT-11. DNA microarray analysis showed that, in p53-deficient cells, the cell cycle regulatory machinery did not respond to CPT-11, leading to the accumulation of the G2/M CDK1/cyclin B complex. Subsequent p53-independent activation of the cdk-inhibitor p21WAF1/CIP1 was observed, which prevented cell cycle progression. Studies by the applicant surprisingly demonstrated that CDK1 induction in p53-deficient cells can be exploited to improve the sensitivity to CPT-11 by additional treatment with a cdk-inhibitor, such as roscovitine. Accordingly, a gain in sensitivity to CPT-11 in a p53 mutated colon cancer cell line can be achieved by restoring wild-type p53 function or by additional treatment with a cdk-inhibitor.

In another particularly preferred embodiment, the invention relates to the use of the combination described herein in the treatment of a CDK dependent or sensitive disorder. CDK dependent disorders are associated with an above normal level of activity of one or more CDK enzymes. Such disorders preferably associated with an abnormal level of activity of CDK2 and/or CDK4. A CDK sensitive disorder is a disorder in which an aberration in the CDK level is not the primary cause, but is downstream of the primary metabolic aberration. In such scenarios, CDK2 and/or CDK4 can be said to be part of the sensitive metabolic pathway and CDK inhibitors may therefore be active in treating such disorders. Such disorders are preferably cancer or leukaemic disorders.

Preferably, the CDK inhibitor is an inhibitor of CDK2 and/or CDK4. More preferably the CDK inhibitor is selected from roscovitine, purvalanol A, purvalanol B, olomucine and other 2,6,9-trisubstituted purines as described in WO97/20842, WO98/05335 (CV Therapeutics), WO99/07705 (Regents of the University of California).

Even more preferably the CDK inhibitor is selected from roscovitine and purvalanol A.

In one particularly preferred embodiment, the CDK inhibitor is roscovitine.

Roscovitine is the compound 2-[(1-ethyl-2-hydroxyethyl)amino]-6-benzylarnine-9-isopropylpurine, also described as 2-(1-D,L-hydroxymethylpropylamino)-6-benzylamine-9-isopropylpurine. As used herein, the term “roscovitine” encompasses the resolved R and S enantiomers, mixtures thereof, and the racemate thereof.

The in vitro activity of roscovitine is as follows:

| Kinase | IC₅₀ (μM) |
|---|---|
| Cdk1/cyclin B | 2.7 |
| Cdk2/cyclin A | 0.7 |
| Cdk2/cyclin E | 0.1 |
| Cdk7/cyclin H | 0.5 |
| Cdk9/cyclin T1 | 0.8 |
| Cdk4/cyclin D1 | 14.2 |
| ERK-2 | 1.2 |
| PKA | >50 |
| PKC | >50 |

Many anti-cancer agents are given in combination in order to optimise the treatment regime. As mentioned above, CPT-11 has been approved, in combination with 5-FU and the modulator leucovorin, as first-line chemotherapy for patients with metastatic CRC (3, 4). However, to date there has been no suggestion of administering CPT-11 in combination with roscovitine.

Even more preferably, the combination is a synergistic combination comprising roscovitine and CPT-11.

In a preferred embodiment, the combination of CPT-11 and roscovitine produces an enhanced effect as compared to either drug administered alone. The surprising nature of this observation is in contrast to that expected on the basis of the prior art.

In one particularly preferred embodiment of the invention, the combination comprises roscovitine and SN-38, which is the active metabolite of CPT-11.

Another aspect of the invention relates to a combination comprising a CDK inhibitor and a DNA topoisomerase 1 inhibitor.

By way of example, preferred DNA topoisomerase 1 inhibitors include CPT-11, camptothecin, topotecan and lurtotecan.

In vitro data have shown that CPT-11-treated HT-29 cells accumulate the G2/M cdk1/cyclin B complex and that sequential treatment of these cells with CPT-11 followed by CYC202 abrogated cdk1 induction and increased cell killing

Accordingly, another aspect of the invention relates to a method of treating a proliferative disorder in a subject, said method comprising the steps of:
(i) administering a DNA topoisomerase 1 inhibitor in an amount sufficient to cause an increase in CDK1 levels; and
(ii) administering a CDK inhibitor in an amount sufficient to induce apoptosis.

Preferably, for this aspect, the DNA topoisomerase 1 inhibitor is CPT-11.

Preferably, for this aspect, the CDK inhibitor is roscovitine.

Even more preferably, steps (i) and (ii) are sequential, i.e. the DNA topoisomerase 1 inhibitor is administered separately or sequentially with respect to the CDK inhibitor.

Even more preferably still, the CDK inhibitor is administered separately or sequentially after administration of the DNA topoisomerase 1 inhibitor.

Pharmaceutical Compositions

Although the components of the present invention (including their pharmaceutically acceptable salts, esters and pharmaceutically acceptable solvates) can be administered alone, for human therapy they will generally be administered in admixture with a pharmaceutical carrier, excipient or diluent.

A preferred embodiment of the invention therefore relates to a pharmaceutical composition comprising a CDK inhibitor and CPT-11 admixed with a pharmaceutically acceptable excipient, diluent or carrier. Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The agents of the present invention can be present as salts or esters, in particular pharmaceutically acceptable salts or esters.

Pharmaceutically acceptable salts of the agents of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The invention also includes where appropriate all enantiomers and tautomers of the agents. The man skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

Some of the agents of the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the agents or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C ¹⁴C , ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agents of the present invention and pharmaceutically acceptable salts thereof can generally be prepared by conventional procedures using appropriate isotopic variations of suitable reagents.

Solvates

The present invention also includes solvate forms of the agents of the present invention. The terms used in the claims encompass these forms.

Polymorphs

The invention furthermore relates to agents of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Chemical Derivatives

The invention also relates to combinations which comprise derivatives of the agents. The term “derivative” as used herein includes chemical modification of an agent. Illustrative of such chemical modifications would be replacement of hydrogen by a halo group, an alkyl group, an acyl group or an amino group.

Prodrugs

The invention further includes agents of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredients can be incorporated into a cream consisting of an aqueous emulsion of polyethylene glycols or liquid paraffin. The active ingredients can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific agents employed, the metabolic stability and length of action of that agent, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, or from 2 to 20 mg/kg, more preferably from 0.1 to 1 mg/kg body weight.

As described above, each active component, the CDK inhibitor and CPT-11 are administered in a therapeutically effective amount preferably in the form of a pharmaceutically acceptable composition. These amounts will be familiar to those skilled in the art. By way of guidance, CPT-11 is typically administered intravenously, orally or topically. Intravenous and oral doses typically comprise 250 mg or 500 mg CPT-11 and are administered in accordance to a physicians direction at a total dosage depending on the weight of a patient e.g. orally at 15 mg/kg weekly, maximum dose 1 g/day, or intravenously 12 mg/kg over 4 hours, or 24-49 mg/kg over 24 hours daily for 5 days. Oral dosages are typically administered in capsules, whereas intra-venous administration is generally administered over a number of hours, typically 4 hours.

Preferably, roscovitine is administered as an orally or intravenously at a dosage of from 1 to 5 g/day. CPT-11 is then administered in the manner deemed most suitable at an appropriate dosage as discussed above. In one preferred embodiment, the CPT-11 is administered at least 24 hours after the administration of roscovitine.

Roscovitine is typically administered orally or intravenously at a dosage of from about 0.05 to about 5 g/day, preferably from about 0.5 to about 5 g/day or 1 to about 5 g/day, and even more preferably from about 1 to about 3 g/day. Alternatively, roscovitine is preferably administered at a dosage of about 0.4 to about 3 g/day. Roscovitine is preferably administered orally in tablets or capsules. The total daily dose of roscovitine can be administered as a single dose or divided into separate dosages administered two, three or four time a day.

The present invention is further described by way of example and with reference to the following figures wherein:

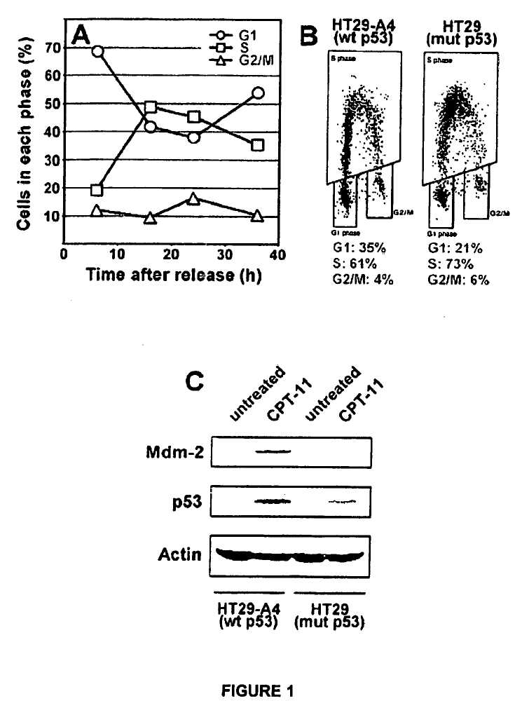

FIG. 1 shows p53 activation and cell cycle blockage in response to CPT-11. In more detail, Mut-p53 HT29 and wt-p53 HT29-A4 cells were synchronized in G0/G1 by serum starvation for 48 h. (A) Histogram showing the cell cycle progression from G1 to S phase and through G2/M transition after release from the block, in untreated control HT29/HT29-A4 cells. (B) BrdU incorporation and cell cycle distribution in synchronized wt-p53 HT29-A4 and mut-p53 HT29 treated cells. 1 μM CPT-11 was added to the medium 6 h after release from G0/G1 and cells were incubated up to 30 h. Note that untreated cells have already completed one cycle and have progressed into the next G1 at 36 h after release. (C) Western blot analysis of synchronized untreated and CPT-11 treated cells, demonstrating the p53 stabilization and activation in wt-p53 HT29-A4 treated cells, in comparison to mut-p53 HT29 treated cells. Synchronized cells were incubated for 24 hours with and without 1 [M CPT-11. Mdm-2 expression was used as a marker of p53 activation; actin is shown as loading control.

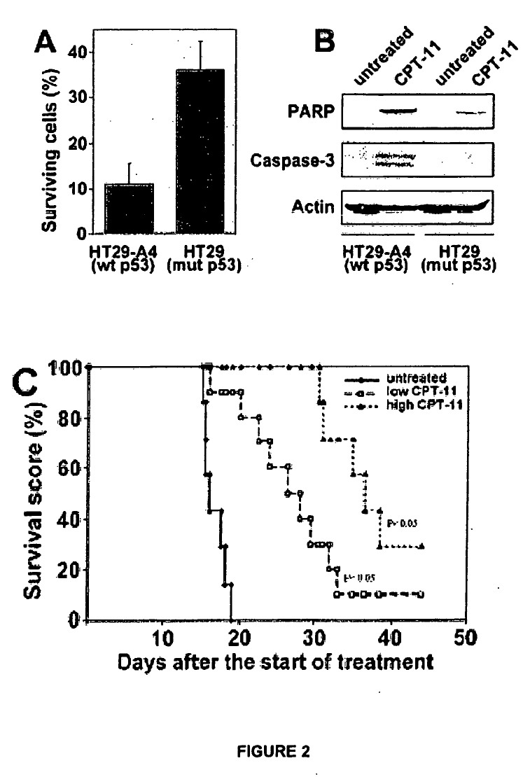

FIG. 2 shows stable expression of wt p53 sensitizes HT29 cells to CPT-11. In more detail, FIG. 2 shows (A) Cell proliferation assays in synchronized wt-p53 HT29-A4 and mut-p53 HT29 cells incubated with 1 μM CPT-11 for 72 h. Results are shown as percentage of cell survival in relation to untreated cells incubated with fresh medium for 72 h. (B) Western blot analysis of cells processed as in (A), demonstrating the significantly higher induction of apoptosis in wtp53 HT29-A4 treated cells, in comparison to mut-p53 HT29 treated cells. PARP and caspase-3 cleaved products were used as markers of apoptosis. (C) Antitumoral effect of CPT-11 (10 and 40 mg/kg/day) in mice bearing HT29-A4 xenografts. The cumulative percentage of individual tumors (mice) reaching a volume of 2,000 mm³ (ethical sacrifice) is plotted as a function of time, corresponding to the survival time of mice.

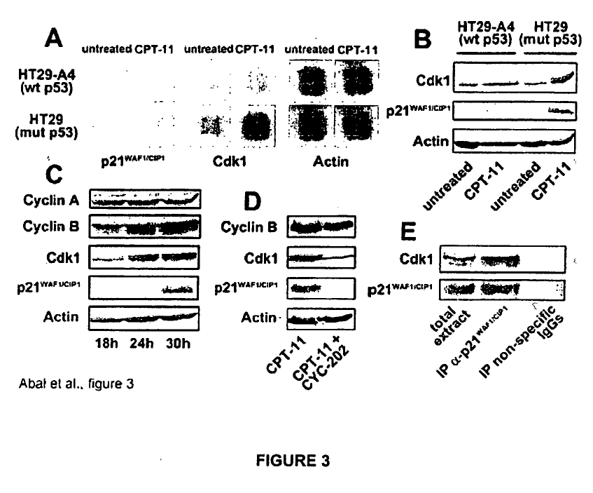

FIG. 3 shows p21^{WAF1/CIP1} and cdk1 activation in response to CPT-11 in mut-p53 HT29 cells. In more detail, FIG. 3 shows (A) Profiles of the expression pattern of p21^{WAF1/CIP1} and cdk1 in wt-p53 HT29-A4 and mutp53 HT29 cells. Six hours after release from G0/G1 block, synchronized cells were incubated for a further 24 h with or without 1 μM CPT-11 and processed for RNA extraction, cDNA synthesis and hybridization on Human Cell Cycle GEArray Q series membranes (see Materials and Methods). The tetra-spots shown are normalized signals of the corresponding genes expression profile. (B) Western blot analysis of p21^{WAF1/CIP1} and cdk1 protein expression in synchronized wt-p53 HT29-A4 and mut-p53 HT29 cells, treated with and without 1 μM CPT-11 under the same conditions as in (A). (C) Time-course of cyclin A, cyclin B, p21^{WAF1/CIP1} and cdk1 protein expression in mut-p53 HT29 cells. Six hours after release from the G0/G1 block, synchronized cells were further incubated with 1 μM CPT-11 for 18, 24, and 30 h. Note that the expression of the cdk1/cyclin B complex correlated with p21^{WAF1/CIP1} activation. (D) p21^{WAF1/CIP1} induction is abolished after incubation with the cdk-inhibitor roscovitine (CYC-202). Six hours after release from G0/G1, synchronized mut-p53 HT29 cells were incubated in the presence of 1 μM CPT-11 for 24 h (first lane), and further incubated with CPT-11 plus 10 μM roscovitine for 24 h (second lane). p21^{WAF1/CIP1} induction, correlating with that of cdk1 after CPT-11 treatment, was abolished when cdk1 was specifically inhibited. (E) Specific co-immunoprecipitation of p21^{WAF1/CIP1} and cdk1 in mut-p53 HT29 cells processed as in (C).

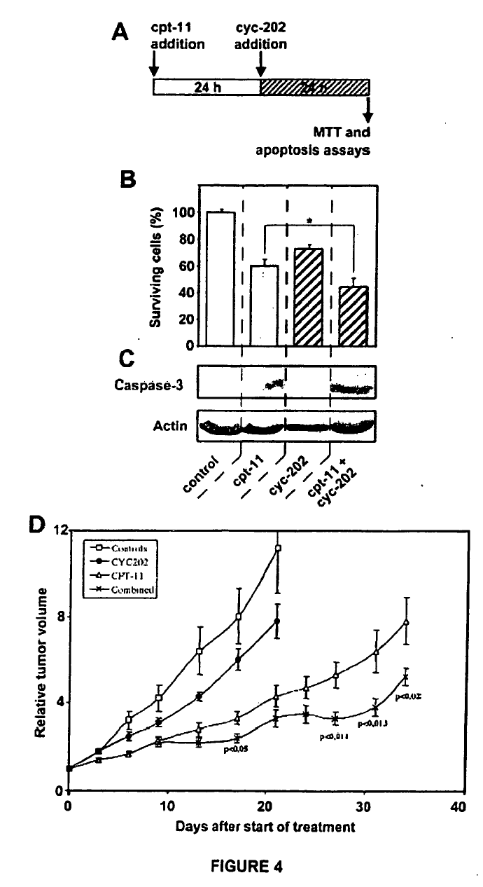

FIG. 4 shows the enhanced sensitivity to CPT-11 of p53-deficient HT29 cells by additional administration of roscovitine. (A) mut-p53 HT29 cells were treated with 1 μM CPT-11 for 24 h; then 10 μM roscovitine was then added to the medium and cells were further incubated for a maximum of 24 h. (B) Histogram showing a decrease in the percentage of mut-p53 HT29 cell survival after additional incubation (slashed-shadowed bar), in comparison to single treatment during 48 h with CPT-11 alone (shadowed bar) or roscovitine alone (slashed bar). (C) Western blot analysis of caspase-3 cleavage in mut-p53 HT29 cells processed as in (B), showing an increased apoptosis induction after additional treatment, in comparison with CPT-11 or roscovitine treatment alone. *p<0.01. (D) Effect of CPT-11 and roscovitine alone or in combination on the growth of HT29 colon tumor xenografts in nude mice. CPT-11 and roscovitine were administered as indicated in Materials and Methods. P values indicate a significant difference between CPT-11 ad the CPT-11+roscovitine combination.

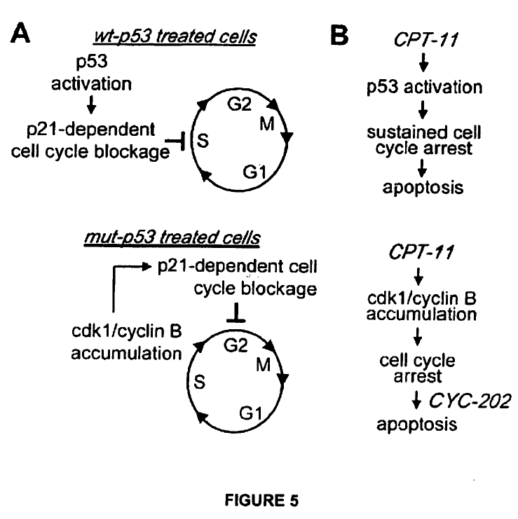

FIG. 5 shows (A) Schematic representation of the response to CPT-11 in wt-p53 and mut-p53 HT29 cells. CPT-11 induced DNA damage activates a p53-dependent response in HT29-A4 cells, resulting in p21^{WAF1/CIP1} induction leading to cell cycle arrest, and eventually triggering apoptosis as a result of a sustained blockage. In mut-p53 HT29 cells, no p53-dependent response is activated after CPT-11 induced DNA damage, and the cell cycle regulatory machinery progresses through S phase; p21^{WAF1/CIP1} is then activated to inhibit the accumulated cdk1/cyclin B complex, thus preventing cell progression into G2/mitosis. (B) Sensitivity to CPT-11 in wt-p53 and mut-p53 HT29 cells. Activation of p53 in response to CPT-11 eventually leads to apoptosis as a result of a sustained cell cycle arrest. In mut-p53 cells, the additional incubation with roscovitine exploits the accumulation of cdk1/cyclin B complexes to improve the sensitivity to CPT-11, by inducing arrested cells into apoptosis.

### EXAMPLES

The abbreviations used herein are as follows: CPT, camptothecin; CRC, colorectal cancer; 5-FU, 5-fluorouracil; CDK, cyclin-dependent kinase; PARP, poly ADP-ribose polymerase; SCF, Skp1-cullin-Fbox; APC/C, anaphase promoting complex/cyclosome; CDK-I, cyclin-dependent kinase inhibitor; APC, adenomatous polyposis coli; TGT, tumor growth time; TGI, tumor growth inhibition; PR, partial response.

Materials and Methods

Drugs

CPT-11 (Campto®, Irinotecan) was kindly supplied by Aventis (Vitry sur Seine, France), and roscovitine (hereinafter referred to as “CYC202”) from Cyclacel (Dundee, UK).

Cell Lines

HT29 (mutated p53 in Ala 273 codon) cell line was derived from a sigmoid colon cancer of stage B1, and its subclone HT29-A4, transfected with a wt p53 expression vector (13). The transfected wt p53 had a dominant function of in the HT29-A4 cell line, as shown previously for HT29-A3 cell line (unpublished data; (14)). Cells were maintained in DMEM medium supplemented with 10% fetal calf serum, and under continuous selection with geneticin for the wt-p53 HT29-A4 cell line.

Cell Cycle Progression and Cell Proliferation

Cells were synchronized in G0/G1 by serum starvation for 48 h. Addition of serum and fresh medium released cells from the block and promoted them to cycle. Cell cycle progression was analyzed by BrdU (5-bromo-2′-deoxiuridine; Sigma) incorporation as described (13). Cell proliferation was measured using the MTT (3-[4,5-dimethylthiazol-2-yl]-2,5-diphenyltetrazolium bromide; Sigma) colorimetric reduction method, as indicated (15).

DNA Microarrays

The profile expression of a panel 96 genes involved in the cell cycle regulation was performed with the Human Cell-cycle GEArray Q series (SuperArray, Inc., Bethesda, Md.). Total RNA was isolated with a RNA purification kit (Quiagen, Valencia, Calif.), and used as the template for 32P-labelled cDNA probe synthesis, according to manufacture's instructions. Hybridization was performed overnight at 60° C. on a nylon membrane printed with tetra-spots of gene-specific cDNA fragments of the 96 cell cycle regulation genes. After extensive washing with SSC (saline-sodium citrate) and SDS (sodium-dodecyl sulfate) buffers (2×SSC/1% DSD; 0.1×SSC/0.5 SDS), digital images of the membranes were obtained with a phosphorimager, and quantified with ImageQuant software (SuperArray).

Immuno-Precipitation and Western-Blot Analysis

Synchronized mut-p53 HT29 cells incubated for 24 hours in the presence of 1 μM CPT-11 were lysed in RIPA buffer (Tris 50 mM, pH 8, NaCl 150 mM, NP40 1%, DOC 0.5%, SDS 0.1%, protease inhibitors). The supernatant was pre-incubated with protein-A sepharose (Amersham Bioscience, Sweden) before overnight incubation at 4° C. with 5 μg of monoclonal p21^{WAF1/CIP1} antibodies (Becton Dickinson, Franklin Lakes, N.J.). Protein-A sepharose beads were used to isolate the target proteins coupled to the antibodies. After extensive washing in lysis buffer, the beads were resuspended and boiled in Laemmli buffer, and processed for western blot analysis. Alternatively, cells were lysed and the supernatant boiled in Laemmli buffer. Samples were resolved on 10-12% SDS-PAGE gels, transferred onto a nitrocellulose membrane (Schleicher & Schuell, France), and incubated with primary antibodies (1:2000 mAb ∝-actin, Sigma; 1:50 mAb ∝-p53 X77, gift from T. Soussi, Curie, France; 2 μg/ml mAb ∝-mdm-2, Oncogene Research Products, San Diego, Calif.; 1:100 pAb ∝-PARP p85 fragment, Promega, Madison, Wis.; 1:1000 pAb ∝-cleaved caspase-3, Cell Signaling Technology, Beverly, Mass.; 1:2500 mAb ∝-cdk1, BD Transduction Laboratories, San Jose, Calif.; 1:400 p Ab ∝-p21WAF1/CIP1, Becton Dickinson; 1:500 pAb ∝-cyclin B and 1:500 pAb ∝-cyclin A; Sigma). Proteins were revealed with alkaline phosphatase conjugate antibodies (1:7500, Promega) and the NBT/BCIP color development substrates (Promega).

Monitoring Apoptosis

Suitable methods for monitoring apoptosis include one or more of the following: flow cytometry, analysis of PARP cleavage, analysis of Caspase cleavage, TUNEL staining, Annexin V labelling, M30 labelling and analysis of proteomic profiles by SELDI-TOF mass spectrometry.

In Vivo Experiments

Swiss nu/nu mice of 6-15 week old (25-35 g/body wt) were bred in the animal facilities of the Curie Institut, Paris (France), and maintained under specified pathogen-free conditions. Their care and housing were in accordance with the institutional guidelines of the French Ethical Committee (Ministère de l'Agriculture et de la Pêche, Ministère de la Recherche, France) and under the supervision of authorized investigators. HT29 and HT29-A4 cell lines were established as transplantable tumors by subcutaneous injection of 2×106 cells. Randomized mice carrying subcutaneous grafts of 60-250 mm³ tumor fragments were treated with CPT-11 administered i.p. in a 0.2 ml volume using an intermittent schedule: two doses were tested, a high total dose of 240 mg/kg (6 injections of 40 mg/kg/day every 4 days) and a low total dose of 40 mg/kg (4 injections of 10 mg/kg/day every 4 days). Mice in the control groups received 0.2 ml of the drug-formulating vehicle. The tumor volume (V=A×B2, where A is the width of the tumor in millimeters and B the length) was measured every 3 days, and tumor growth was calculated as described (15). Combination of CPT-11 with roscovitine was performed by sequential cycles of CPT-11 i.p. at 40mg/kg every seven days, followed by 3-4 consecutive administration of roscovitine twice daily by oral gavage at 200 mg/kg.

Dose Administered

For oral gavage CYC202 was reconstituted prior to each session in 50 mM HCl. It was sonicated for approx. 5 min. after addition of the vehicle and stirred with a magnetic stirrer during the dosing session. Solutions were prepared as shown in the table below with final concentrations between 1-10 mg/ml by adding appropriate amounts of HCl in bottles with pre-weighted CYC202.

| Dose in mg/ml | Volume ml | Drug in bottle | Addition of 50 mM HCl |
|---|---|---|---|
| 1 | 300 | 300 mg | 300 ml |
| 5 | 300 | 1500 | 298 ml |
| 7.5 | 200 | 1500 | 198 ml |
| 10 | 150 | 1500 | 148 ml |

Sequence and Duration of Administration

When given alone, CYC202 was administered twice daily (2×200 mg/kg or 2×150 mg/kg/d) at ˜8h intervals (˜ at 10 am and ˜6 pm) for the same duration as the groups receiving chemotherapy. Mice treated with CYC202 were not fed during the 8 h period of administration, so as not to interfere with the product bioavailability.

When given as combined with chemotherapy in animals with xenografts, CYC202 was given as follows: CYC202 (2×200 mg/kg/day) day 2-5, 9-12, 16-19, CPT-11 day 1, 8, 15

Route of Administration

CYC202 was administered by oral gavage, in a volume of 100 to 200 Ill/mouse. CPT-11 solution was administered by IP route. The volume of injection of individual compounds was from 100 to 200 μl/mouse.

Tumour Transplantation

Tumour xenografts were maintained by serial transplantation into immunodeficient mice. For drug efficacy assays, mice received subcutaneous grafts of tumour in fragments originated from a previous passage. Fragments for this assay originated from 5 donor mice bearing the previous tumour passage and sacrificed when the tumours reached 12 to 15 mm of diameter. All mice from the same experiment were implanted on the same day. At least 10 mice per group were included. Tumour-bearing donor mice were sacrificed by cervical dislocation. The tumour was aseptically excised. Tumours were deposited in a Petri dish containing a culture medium and dissected carefully to remove the fibrous capsule usually surrounding the tumour. Necrotic tumours were rejected. Tumour tissue was kept in culture medium during the transplantation procedure. Xenografts were performed aseptically. After anaesthesia with avertine, and sterilisation of the skin with a 70% alcohol/water solution, the skin was incised at the level of the scapular region, and a tumour fragment was placed in the subcutaneous tissue. Skin was closed with clips.

Evaluation of Antitumor Efficacy

All raw data were manually registered in a book and transferred into a computer. Relative tumour volume (RTV): was calculated as the ratio of the volume at the time t divided by the initial volume at day 1 and multiplied by 100. These data allow to rapidly evaluate the lack of growth when the RTV is equal or under 100% (tumour regressions). Curves of mean RTV as a function of time in treated and control groups were obtained and presented in the report. Optimal growth inhibition: was calculated as the ratio of RTV (×100) in the treated group divided by the RTV in the controls. Growth delay: was calculated as the time in days necessary to multiply by 5 (or 4) an initial tumour volume of 200-400 mm3 in treated and control groups. Mice were individually weighted once a week. Weight variations as compared to initial weight and means (or median) per group were calculated.

Statistics

All statistical tests were performed using Statview software. The following parameters were compared: tumour volume and/or RTV, optimal growth inhibition, growth delay, body weight change. Statistical analysis of the efficacy of the treatment was performed by paired t-test.

Results

Activation of Stably Transfected wt-p53 Blocks Cell Cycle Progression in CPT-11 Treated HT29 Cells

To characterize the effects of CPT-11 on cell cycle progression, specifically on S phase, where this top1 inhibitor exerts its action, cells were synchronized in G0/G1 by serum starvation (see Materials and Methods). Six hours after serum-induced release from the block, when cells started to cycle, CPT-11 at 1 μM concentration that effectively blocks synchronized HT29 cells (not shown), was added to the medium and progression through the cell cycle was analyzed in wt-p53 and mut-p53 treated cells. Untreated cells of both types progressed into S phase around 16 h, through G2/M at 24 h, and completed a cell cycle approximately 36 h after release from the block (FIG. 1A). When incubated up to 36 h in the presence of CPT-11, both wt-p53 and mut-p53 cells were blocked in S phase (FIG. 1B). Interestingly, wt-p53 cells were blocked in early S phase, in contrast to mut-p53 cells, arrested in late S phase (FIG. 1B). This suggested that in wt p53 HT29-A4, cell cycle arrest resulted from the immediate stabilization and activation of wt p53 in response to CPT-11. The induction of p53 was further confirmed by western-blot in CPT-11 treated versus untreated wt-p53 cells (FIG. 1C). p53 protein levels were also slightly increased after CPT-11 treatment in mut-p53 cells, probably reflecting the increase of the mutated inactive form of p53 (FIG. 1C). The functionality of the induced p53 was further analysed by testing the induction of the downstream p53 gene mdm2. Mdm2 protein binds to p53 and acts as its major cellular antagonist, ubiquitinating p53 and addressing it to degradation by the proteasome. Moreover, the mdm2 gene is a direct target for positive transcriptional activation by p53, thus defining the basal p53/mdm2 auto-regulatory loop that results in continuous repression of p53 activity and its maintenance in a biologically inert state (16). mdm2 was observed to be induced in CPT-11 treated wt-p53 cells (FIG. 1C). From these results we conclude that in response to CPT-11, the stably transfected wt p53 was activated and promoted a p53-dependent cell cycle arrest.

In Vitro Enhanced Sensitivity to CPT-11 of wt-p53 Versus mut-p53 HT29 Cells

Studies were undertaken to investigate whether dominant expression of wt p53 influenced the sensitivity of this colon cancer cell line to CPT-11. As shown by cell proliferation assays, incubation of wt-p53 cells with 1 μM CPT-11 for 72 h significantly decreased cell proliferation with respect to untreated cells and in comparison to mut-p53 cells (FIG. 2A). This suggested that, in addition to the cell cycle arrest due to the activation of the stably transfected p53, CPT-11 treatment was associated with an increased apoptosis in wt-p53 cells. This was further confirmed by western blot analysis of the p85 fragment of PARP (poly ADP-ribose polymerase) that results from the caspase cleavage of the 116 kDa intact protein (17) (FIG. 2B). Moreover, the activation of caspase-3, one of the main executers of apoptosis and responsible for PARP cleavage, by proteolytic processing into activated p17 and p12 subunits, was confirmed in wtp53 cells treated with CPT-11, in comparison to mut-p53 cells (FIG. 2B).

In Vivo Enhanced Sensitivity to CPT-11 of wt-p53 Versus mut-p53 HT29 Cells

The response to CPT-11 in wt-p53 and mut-p53 was compared using established xenografts. Both high and low CPT-11 concentrations inhibited the growth of wt-p53 xenografts with a mean TGI of 60% and a mean TGT of 18 (±7) and 87% and a mean TGT of 30 (±7), respectively (Table 1). Moreover, CPT-11 treatment at both doses significantly increased the survival score of mice bearing the wt-p53 HT29-A4 hCRC xenografts, shown as the cumulative percentage of individual tumors (mice) reaching a volume of 2000 mm³ (ethical sacrifice) as a function of time (FIG. 2C). In contrast, no response was observed in the mut-p53 xenografts (not shown). These results indicate that, at least in the colon cancer cell model used, the presence of a functional p53 significantly improved the response to CPT-11.

Involvement of p21^{WAF1/CIP1} in CPT-11 Induced Cell Cycle Arrest

To understand the mechanism underlying cell cycle arrest in response to CPT-11 in the p53-deficient tumor cells, DNA microarray technology (SuperArray) was used to analyse the expression profile of a panel of 96 genes involved in the cell cycle regulation. These arrays are focused on cyclin-dependent kinase (cdk) genes as main regulators of cell cycle, and genes regulating the activities of cdks at multiple levels, such as cyclins, cdk inhibitors, cdk phosphatases, and cdk kinases. In addition, they include genes essential for DNA damage and mitotic spindle checkpoints, as well as genes in the SCF (Skp1-cullin-Fbox) and APC/C (anaphase promoting complex/cyclosome) ubiquitin-conjugation complexes. A slight activation of p21^{WAF1/CIP1} was observed in both wt-p53 and mut-p53 cell lines treated with 1 μM CPT-11 (FIG. 3A). Western blot analysis was performed to confirm the DNA-microarray indication that p21^{WAF1/CIP1} was involved in the response to CPT-11. The faint transcriptional activation was observed to correspond to a significantly higher induction of p21^{WAF1/CIP1} protein levels in the mut-p53 treated cells, compared to those of wt-p53 treated cells (FIG. 3B). This suggests that the S-phase cell cycle arrest, observed when both wt-p53 and mut-p53 cell lines were incubated in the presence of CPT-11, resulted from the induction of p21^{WAF1/CIP1}.

Induction of Cdk1 in CPT-11 Treated p53-Deficient Cells

DNA-microarray analysis showed that the most significant difference in expression among untreated and CPT-11 treated wt-p53 cells versus mut-p53 cells, corresponded to cdc2/cdk1, the kinase responsible for cell cycle progression through S phase to G2/Mitosis (FIG. 3A). Western blot analysis confirmed the increased levels of cdk1 in mut-p53 cells treated with 1 μM CPT-11 (FIG. 3B). Cdks form complexes with their respective cyclins depending on the phase of the cell cycle, the cyclin being the regulatory unit and the cdk the catalytic partner. Studies were undertaken to investigate whether the increased cdk1 expression correlated with the expression of one of its corresponding cyclins: the cyclin A, known to complex cdk1 in late S/G2 phase, or the cyclin B, involved in the G2/M transition. The kinetics of cdk1 expression in mut-p53 cells treated with CPT-11 paralleled those of cyclin B but not cyclin A (FIG. 3C). This indicates that, while cells were blocked in S phase, their cell cycle regulators corresponded to that of the G2/M transition. Interestingly, it was also observed that the increase in cdk1/cyclin B expression occurred before that of p21WAF1/CIP1, known to block cell cycle progression by acting as a potent cdk inhibitor (FIG. 3C). Moreover, p21^{WAF1/CIP1} induction was completely abolished when CPT-11 treated mut-p53 cells were further incubated with roscovitine, a potent and selective cdk-inhibitor (cdk-I) (18). High cdk1 levels after incubation with 1 μM CPT-11 (FIG. 3D, first lane) were inhibited by further treatment with CPT-11 plus 10 μM roscovitine (FIG. 3D, second lane). Inhibition of p21^{WAF1/CIP1} correlated with that of cdk1, suggesting that p21^{WAF1/CIP1} was induced in response to the accumulation of the cdk1/cyclin B complex, after treatment with CPT-11 in mut-p53 cells. This was confirmed by co-immunoprecipitation of cdk1 with an antibody directed against p21WAF1/CIP1, in mut-p53 cells incubated during 24 h with 1 μM CPT-11 (FIG. 3E). This indicates that the response to the high levels of cdk1 was counteracted by p21^{WAF1/CIP1} induction, interaction that is known to promote cdk1 inhibition thus preventing cells from progression into G2/M (19).

Enhanced Sensitivity of p53-deficient HT29 Cells to CPT-11 after Additional Treatment with Roscovitine

The characterization of the molecular mechanisms underlying the response to CPT-11 in p53-mut HT29 cells, prompted efforts to improve their sensitivity to CPT-11 by exploiting the accumulation of cdk1 after 24 h of treatment, and designing an additional incubation with the specific cdk-I roscovitine. Mut-p53 HT29 cells were initially pre-treated with 1 μM CPT-11 for 24 h; then roscovitine was added to the medium and cells were further incubated to a maximum of 24 h in the presence of both drugs (FIG. 4A). Untreated cells, cells treated for 48 h with CPT-11 alone and with roscovitine alone for 24 h were used as controls. Treatment with roscovitine alone promoted cell cycle blockage (FIG. 4B). In contrast, addition of roscovitine to cells that have accumulated cdk1 by pre-treatment with CPT-11 resulted in a significant decrease in cell proliferation when compared with CPT-11 alone (FIG. 4B). Western-blot analysis of caspase-3 cleavage showed an increased apoptosis after the additional treatment with roscovitine, in comparison to roscovitine or CPT-11 alone (FIG. 4C). This hypothesis was also analysed in vivo, by sequential administration of CPT-11 and roscovitine in nude mice bearing HT29 tumor xenografts. Tumor growth delay (TGD; days to reach a 4-fold increase in tumor volume from the size at the start of treatment) showed an additive/synergistic effect of the combination between CPT-11 and roscovitine (TGD±SD: 30±1,7), when compared to CPT-11 alone (22±1,9), roscovitine alone (12±1,2) or control untreated (9±1) (FIG. 4D). These results suggest that a combination of CPT-11 and a cdk-I would be therapeutically effective in the treatment of colorectal cancer, when mutations in p53 diminish the sensitivity to the top1 inhibitor CPT-11.

In Vivo Drug Efficacy Results

The antitumour activity data are summarized in Table 2 and are shown as tumour growth curves (mean relative tumor volume against time). The mean tumor volumes at the start of treatment were 263.4±17.6 mm³ for the HT29 colon adenocarcinoma.

At the doses selected from prior tolerance studies, CYC202 was well tolerated, alone or in combination with chemotherapy, for three to six 4-day cycles. CYC202 at 400 mg/kg/d was well tolerated in combination with CPT-11 at 40 mg/kg in the HT-29 model.

CYC202 as Single Agent

CYC202 did not show antitumour activity the xenograft model when given at the maximum tolerated dose of 400 mg/kg/day for up to six weeks.

CYC202 in Combination with Chemotherapy

The combination of CYC202 with CPT-11 gave significant results, with a probable synergy observed in the HT29 colon carcinoma. The effect of the association was statistically significant in terms of both TGD and TGI (see Table 2 and FIG. 4D).

Discussion

CRC is considered the paradigm of the multistep progression cancer model, where genetic alterations accompany tumorigenesis (20), although alternative genetic pathways may contribute to the progression of the disease (21, 22). Crucial molecular events involve alteration and mutation of adenomatous polyposis coli (APC) and Kirsten-ras (K-ras) genes. In addition, mutations in the tumor-suppressor gene p53 appear to be a late phenomenon in CRC, which may allow the growing tumor with multiple genetic alterations to evade cell cycle arrest and apoptosis (23, 24). Nowadays, it is largely assumed that in addition to being directly responsible for the antitumor effect, agents damaging DNA may initiate post-damage responses by activating cell-cycle checkpoints (25). Accordingly, the integrity of these damage responses might also influence treatment sensitivity, and disabling apoptotic pathways activated by anticancer agents may contribute to resistance (26).

The above studies reveal that CPT-11 treatment in G0/G1 synchronised HT29 cells transfected with wt p53, resulted in a functionally active p53 leading to cell cycle arrest in S-phase. It was also observed that the presence of a functional p53 correlated in vitro with an increased apoptosis and in vivo with an improved sensitivity, in response to CPT-11. These results are in agreement with those obtained in a comparative study between wt/mut p53 hCRC xenografts, that showed that mutated p53 correlated with a poor response to CPT-11(10).

The analysis of the expression profile of a panel of genes involved in the cell cycle regulation, showed that the S-phase cell cycle arrest induced by CPT-11 resulted from the induction of p21^{WAF1/CIP1} p21^{WAF1/CIP1} is found in a complex involving cyclins and cdks and appears to be a universal inhibitor of cdk activity. Induction of p21^{WAF1/CIP1} can occur in response to DNA damage or mitogenic stimuli and even during differentiation, both in a p53-dependent and independent upregulation. Upregulation of p21^{WAF1/CIP1} has been related to both p53-dependent and independent apoptosis in breast cancer after CPT treatment (27), and the Fas pathway and ceramide signaling have been implicated in the p53-independent induction of apoptosis by camptothecin-treated mut-p53 HT29 cells (28, 29). Further studies have demonstrated that in mut-p53 HT29 cells treated with CPT-11, p21 induction depends on a specific accumulation of the G2/M cyclin-dependent kinase cdk1. Unscheduled activation of cdk1 has been observed in CPT-11 treated human promyelocytic leukemia HL60 cells prior to apoptosis (30). All these results lead to the proposal of two mechanisms to account for the response to CPT-11, in wt-p53 and in mut-p53 conditions (FIG. 5A). Firstly, a functional p53 is capable of triggering a direct response to CPT-11, probably by activating the DNA damage checkpoint in response to the DNA double-strand breaks. This would involve the activation of the downstream upregulated p21^{WAF1/CIP1}, eventually promoting apoptosis as a result of a sustained blockage. Secondly, in mutated p53 cells, CPT-11 imposes an arrest in cell cycle progression during S phase, probably due to the inability of cells to successfully complete DNA synthesis. Nevertheless, in the absence of a functional p53 activating the response to CPT-11, the cell cycle machinery continues to progress and to accumulate cdk1/cyclin B complexes; p21_{WAF1/CIP1} is then induced in a p53-independent manner to suppress cdk1 activity and to protect cells from progression into G2/M.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

TABLE 1
Antitumor effects of low dose (10 mg/kg/day) or high dose
(40 mg/kg/day) CPT-11 on wt-p53 HT29-A4 xenografts

| | | Growth | TGI, | | | |
|---|---|---|---|---|---|---|
| Treatment | Mean TGT^{a} | delay^{b}, | %^{c} | No. mice | No. | No. |
| (mg/kd/d) | (SD), days | days | (days) | per group | PR^{d} | cures^{e} |
| 0 | 8 (2) | / | / | 7 | / | / |
| 10 | 18* (7) | 10 | 60 | 10 | 8 | / |
| 40 | 30* (7) | 22 | 86 | 8 | 7 | 1 |

^{a}Tumor growth time: time in days necessary to reach a five-fold increase of individual tumor volume from the size at the start of treatment (60-250 mm³);
^{b}Growth delay: calculated as the difference between treated/control TGT;
^{c}Tumor growth inhibition: calculated as the ratio between treated and control growth inhibition, at the date of the ethical sacrifice of the first control mouse bearing a tumor with a volume of 2000 mm³;
^{d}Partial response: up to 50% of individual growth inhibition;
^{e}Tumor-free mice were defined as mice without any palpable tumor at the end of the experiment.
The 2 paired Student's t test was used. *Significantly different from control group: 10 − 3 < p < 10 − 6.

TABLE 2
In vivo drug efficacy data

| | Mean (SD) | Calc growth | Calc combo | TGI % | Fraction | Calc combo | Mice | PR^{g} | CR^{h} | Curesⁱ |
|---|---|---|---|---|---|---|---|---|---|---|
| Treatment | TGD^{a} | delay^{b} | growth delay^{c} | (day)^{d} | T/C^{e} | effect^{f} | nb | nb | nb | nb |
| Control | 9.3 | | | | | | 9 | | | |
| | (1.0) | | | | | | | | | |
| CYC202 | 12.3 | 3.0 | | 30.4 | 69.6 | | 9 | 0 | 0 | 0 |
| (400 mg/kg) | (1.2) | | | (21) | | | | | | |
| CPT-11 | 21.9¹ | 12.6 | | 61.6 | 38.4 | | 9 | 0 | 0 | 0 |
| (20 mg/kg) | (1.2) | | | (21) | | | | | | |
| CPT-11 + | 29.6² | 20.3 | 15.6 | 70.5 | 29.5 | 26.7 | 9 | 0 | 0 | 0 |
| CYC202 | (1.7) | | | (21) | | | | | | |

¹significantly different from control group at p < 0.01.
²significantly different from single agent group at p < 0.02
^{a}Tumour growth delay: days to reach a 4-fold increase of individual tumour volume from the size at the start of treatment.
^{b}Calc. growth delay: TGD treated − TGD control group.
^{c}Sum of calc. growth delays for single agents.
^{d}Tumour growth inhibition: (control RTV-treated RTV)/control RTV × 100 at indicated day.
^{e}Treated RTV/control RTV × 100.
^{f}Fraction T/C single agent #1 × fraction T/C single agent #2.
^{g}Partial response: at least 50% of individual tumour regression at any time after start of treatment.
^{h}CR: complete regression.
ⁱCure: tumour-free mice defined as bearing no palpable tumour at the end of the experiment.

### REFERENCES

1. Fishman, A. D. and Wadler, S. Advances in the treatment of metastatic colorectal cancer. Clin. Colorectal Cancer, 1: 20-35, 2001.
2. Sobrero, A., Kerr, D., Glimelius, B., Van Cutsem, E., Milano, G., Pritchard, D. M., Rougier, P., and Aapro, M. New directions in the treatment of colorectal cancer: a look to the future. Eur. J. Cancer, 36: 559-66, 2000.
3. Rougier, P. and Mitry, E. Review of the role of CPT-11 in the treatment of colorectal cancer, Clin. Colorectal Cancer, 1: 87-94, 2001.
4. Vanhoefer, U., Harstrick, A., Achterrath, W., Cao, S., Seeber, S., and Rustum, Y. M. Irinotecan in the treatment of colorectal cancer: clinical overview. J. Clin. Oncol., 19:1501-18, 2001.
5. Tanizawa, A., Fujimori, A., Fujimori, Y., and Pommier, Y. Comparison of topoisomerase I inhibition, DNA damage, and cytotoxicity of camptothecin derivatives presently in clinical trials. J. Natl. Cancer Inst., 86: 836-42, 1994.
6. Kjeldsen, E., Svejstrup, J. Q., Gromova, II, Alsner, J., and Westergaard, O. Camptothecin inhibits both the cleavage and religation reactions of eukaryotic DNA topoisomerase I. J. Mol. Biol., 228: 1025-30, 1992.
7. Magrini, R., Bhonde, M. R., Hanski, M. L., Notter, M., Scherubl, H., Boland, C. R., Zeitz, M., and Hanski, C. Cellular effects of CPT-11 on colon carcinoma cells: dependence on p53 and hMLH1 status. Int. J. Cancer, 101: 23-31, 2002.
8. Gupta, M., Fan, S., Zhan, Q., Kohn, K. W., O'Connor, P. M., and Pommier, Y. Inactivation of p53 increases the cytotoxicity of camptothecin in human colon HCT116 and breast MCF-7 cancer cells. Clin. Cancer Res., 3: 1653-60, 1997.
9. Blagosklonny, M. V. and El-Deiry, W. S. Acute overexpression of wt p53 facilitates anticancer drug-induced death of cancer and normal cells. Int. J. Cancer, 75: 933-40, 1998.
10. Bras-Goncalves, R. A., Rosty, C., Laurent-Puig, P., Soulie, P., Dutrillaux, B., and Poupon, M. F. Sensitivity to CPT-11 of xenografted human colorectal cancers as a function of microsatellite instability and p53 status. Br. J. Cancer, 82: 913-23, 2000.
11. Lansiaux, A., Bras-Goncalves, R. A., Rosty, C., Laurent-Puig, P., Poupon, M. F., and Bailly, C. Topoisomerase I-DNA covalent complexes in human colorectal cancer xenografts with different p53 and microsatellite instability status: relation with their sensitivity to CTP-11. Anticancer Res., 21: 471-6, 2001.
12. Xie, X., Sasai, K., Shibuya, K., Tachiiri, S., Nihei, K., Ohnishi, T., and Hiraoka, M. P53 status plays no role in radiosensitizing effects of SN-38, a camptothecin derivative. Cancer Chemother. Pharmacol., 45: 362-8, 2000.
13. Pocard, M., Chevillard, S., Villaudy, J., Poupon, M. F., Dutrillaux, B., and Remvikos, Y. Different p53 mutations produce distinct effects on the ability of colon carcinoma cells to become blocked at the GI/S boundary after irradiation. Oncogene, 12: 875-82, 1996.
14. de Cremoux, P., Salomon, A. V., Liva, S., Dendale, R., Bouchind'homme, B., Martin, E., Sastre-Garau, X., Magdelenat, H., Fourquet, A., and Soussi, T. p53 mutation as a genetic trait of typical medullary breast carcinoma. J. Natl. Cancer Inst., 91: 641-3, 1999.
15. Bras-Goncalves, R. A., Pocard, M., Formento, J. L., Poirson-Bichat, F., De Pinieux, G., Pandrea, I., Arvelo, F., Ronco, G., Villa, P., Coquelle, A., Milano, G., Lesuffleur, T., Dutrillaux, B., and Poupon, M. F. Synergistic efficacy of 3n-butyrate and 5-fluorouracil in human colorectal cancer xenografts via modulation of DNA synthesis. Gastroenterology, 120: 874-88, 2001.
16. Oren, M., Damalas, A., Gottlieb, T., Michael, D., Taplick, J., Leal, J. F., Maya, R., Moas, M., Seger, R., Taya, Y., and Ben-Ze'ev, A. Regulation of p53: intricate loops and delicate balances. Biochem. Pharmacol., 64: 865-71, 2002.
17. Whitacre, C. M., Zborowska, E., Willson, J. K., and Berger, N. A. Detection of poly(ADP-ribose) polymerase cleavage in response to treatment with topoisomerase I inhibitors: a potential surrogate end point to assess treatment effectiveness. Clin. Cancer Res., 5: 665-72, 1999.
18. McClue, S. J., Blake, D., Clarke, R., Cowan, A., Cummings, L., Fischer, P. M., MacKenzie, M., Melville, J., Stewart, K., Wang, S., Zhelev, N., Zheleva, D., and Lane, D. P. In vitro and in vivo antitumor properties of the cyclin dependent kinase inhibitor CYC-202 (R-roscovitine). Int. J. Cancer, 102: 463-8, 2002.
19. Taylor, W. R. and Stark, G. R. Regulation of the G2/M transition by p53. Oncogene, 20: 1803-15, 2001.
20. Fearon, E. R. and Vogelstein, B. A genetic model for colorectal tumorigenesis. Cell, 61: 759-67, 1990.
21. Janssen, K. P., el-Marjou, F., Pinto, D., Sastre, X., Rouillard, D., Fouquet, C., Soussi, T., Louvard, D., and Robine, S. Targeted expression of oncogenic K-ras in intestinal epithelium causes spontaneous tumorigenesis in mice. Gastroenterology, 123: 492-504, 2002.
22. Smith, G., Carey, F. A., Beattie, J., Wilkie, M. J., Lightfoot, T. J., Coxhead, J., Garner, R. C., Steele, R. J., and Wolf, C. R. Mutations in APC, Kirsten-ras, and p53-alternative genetic pathways to colorectal cancer. Proc. Natl. Acad. Sci. U S A, 99: 9433-8, 2002.
23. Arends, J. W. Molecular interactions in the Vogelstein model of colorectal carcinoma. J. Pathol., 190: 412-6, 2000.
24. Gryfe, R., Swallow, C., Bapat, B., Redston, M., Gallinger, S., and Couture, J. Molecular biology of colorectal cancer. Curr. Probl. Cancer, 21: 233-300, 1997.
25. Johnstone, R. W., Ruefli, A. A., and Lowe, S. W. Apoptosis: a link between cancer genetics and chemotherapy. Cell, 108: 153-64, 2002.
26. Lowe, S. W., Ruley, H. E., Jacks, T., and Housman, D. E. p53-dependent apoptosis modulates the cytotoxicity of anticancer agents. Cell, 74: 957-67, 1993.
27. Liu, W. and Zhang, R. Upregulation of p21^{WAF1/CIP1} in human breast cancer cell lines MCF-7 and MDA-MB-468 undergoing apoptosis induced by natural product anticancer drugs 10-hydroxycamptothecin and camptothecin through p53-dependent and independent pathways. Int. J. Oncol., 12: 793-804, 1998.
28. Chauvier, D., Morjani, H., and Manfait, M. Ceramide involvement in homocamptothecin- and camptothecin-induced cytotoxicity and apoptosis in colon T29 cells. Int. J. Oncol., 20: 855-63, 2002.
29. hao, R. G., Cao, C. X., Nieves-Neira, W., Dimanche-Boitrel, M. T., Solary, E., and Pommier, Y. Activation of the Fas pathway independently of Fas ligand during apoptosis induced by camptothecin in p53 mutant human colon carcinoma cells. Oncogene, 20: 1852-9, 2001.
30. Shimizu, T., O'Connor, P. M., Kohn, K. W., and Pommier, Y. Unscheduled activation of cyclin B1/Cdc2 kinase in human promyelocytic leukemia cell line HL60 cells undergoing apoptosis induced by DNA damage. Cancer Res., 55: 228-31, 1995.
31. Motwani, M., Jung, C., Sirotnak, F. M., She, Y., Shah, M. A., Gonen, M., and Schwartz, G. K. Augmentation of apoptosis and tumor regression by flavopiridol in the presence of CPT-11 in Hctl16 colon cancer monolayers and xenografts. Clin. Cancer Res., 7: 4209-19, 2001.
32. Shao, R. G., Cao, C. X., Shimizu, T., O'Connor, P. M., Kohn, K. W., and Pommier, Y. Abrogation of an S-phase checkpoint and potentiation of camptothecin cytotoxicity by 7-hydroxystaurosporine (UCN-01) in human cancer cell lines, possibly influenced by p53 function. Cancer Res., 57: 4029-35, 1997.

1. A combination comprising CPT-11 and a CDK inhibitor.

2. The combination according to claim 1 wherein the CDK inhibitor is an inhibitor of CDK2 or CDK4.

3. The combination according to claim 2 wherein the CDK inhibitor is selected from the group consisting of roscovitine, purvalanol A, purvalanol B and olomoucine.

4. The combination according to claim 3 wherein the CDK inhibitor is roscovitine.

5. The combination according to claim 1 wherein the CDK inhibitor is a 2,6,9-trisubstituted purine.

6. A pharmaceutical composition comprising CPT-11, a CDK inhibitor and a pharmaceutically acceptable carrier, diluent or excipient.

7. A pharmaceutical product comprising CPT-11 and CDK inhibitor as a combined preparation for simultaneous, sequential or separate use in therapy.

8. The pharmaceutical product according to claim 7 wherein the CDK inhibitor is an inhibitor of CDK2 or CDK4.

9. The pharmaceutical product according to claim 8 wherein the CDK inhibitor is selected from the group consisting of roscovitine, purvalanol A, purvalanol B and olomoucine.

10. The pharmaceutical product according to claim 9 wherein the CDK inhibitor is roscovitine.

11. The pharmaceutical product according to claim 7 further comprising a pharmaceutically acceptable carrier, diluent or excipient.

12. The pharmaceutical product according to claim 7 wherein the CDK inhibitor is a 2,6,9-trisubstituted purine.

13. A method of treating a proliferative disorder in a subject, said method comprising administering to said subject CPT-11 and a CDK inhibitor.

14. The method according to claim 13, wherein the method comprises administering said CDK inhibitor to said subject prior to administering said CPT-11 to said subject.

15. The method according to claim 13 which comprises administering said CPT-11 to said subject prior to administering said CDK inhibitor to said subject.

16. The method according to claim 13 wherein the CDK inhibitor is an inhibitor of CDK2 or CDK4.

17. The method according to claim 16 wherein the CDK inhibitor is selected from the group consisting of roscovitine, purvalanol A, purvalanol B and olomoucine.

18. The method according to claim 17 wherein the CDK inhibitor is roscovitine.

19. The method of claim: 1 3, wherein the CDK inhibitor is a 2,6,9-trisubstituted purine.

20. The method according to claim 13 wherein the CDK inhibitor and CPT-11 are each administered in a therapeutically effective amount with respect to the individual components.

21. The method according to claim 13 wherein the CDK inhibitor and CPT-11 are each administered in a subtherapeutic amount with respect to the individual components.

22. The method according to claim 13 wherein the proliferative disorder is cancer.

23. The method according to claim 22 wherein the cancer is colorectal cancer or lung cancer.

24. The method of claim 13, wherein the CPT-11 is administered in an amount sufficient to increase CDK1 levels.

25. The method of claim 13, wherein the CDK inhibitor is administered in an amount sufficient to induce apoptosis.

26. The method of claim 13, wherein the CPT-11 and the CDK inhibitor are administered simultaneously.

27. The method of claim 13, wherein the CPT-11 and the CDK inhibitor are administered separately.

28. The method of claim 13, wherein the CPT-11 and the CDK inhibitor are administered sequentially.

29. A method of preparing a pharmaceutical product for the treatment of a proliferative disorder, comprising combining CPT-11 and a CDK inhibitor in a preparation, such that the CPT-11 and the CDK inhibitor may be administered simultaneously, sequentially or separately.

30. A combination comprising a CDK inhibitor and a DNA topoisomerase 1 inhibitor.

31. The combination according to claim 30 wherein the DNA topoisomerase 1 inhibitor is selected from the group consisting of CPT-11, camptothecin, topotecan and lurtotecan.

32. A method of treating a proliferative disorder in a subject, said method comprising the steps of:
(i) administering a DNA topoisomerase 1 inhibitor in an amount sufficient to increase CDK1 levels; and
(ii) administering a CDK inhibitor in an amount sufficient to induce apoptosis.

33. The method according to claim 32 wherein the DNA topoisomerase 1 inhibitor is CPT-11.

34. The method according to claim 32 wherein the CDK inhibitor is roscovitine.

35. The method according to claims 32 wherein steps (i) and (ii) are sequential.
